(19) United States
(12) Patent Application Publication
(10) Pub. No.: US 2005/0159770 A1
(43) Pub. Date: Jul. 21, 2005

(54) Funnel catheter device and method of operation thereof

(75) Inventors: Afshin A. Divani, Encino, CA (US); Adnan I. Qureshi, Orange, NJ (US); Robert LuDuca, Santa Cruz, CA (US)

Correspondence Address: CROWELL & MORING LLP;INTELLECTUAL PROPERTY GROUP, P.O. BOX 14300, WASHINGTON, DC, 20044-4300, US

(73) Assignee: DIQUR MEDICAL SYSTEMS, LLC, Encino (US)

(21) Appl. No.: 10/760,722

(22) Filed: Jan. 21, 2004

(51) Int. Cl. A61M 29/00

(52) U.S. Cl. 606/200

(57) ABSTRACT

A funnel or cone-shaped portion of a funnel device is fixed to the distal end of a guide catheter. The funnel is configured with an adjustable opening. The funnel can be fixed to a guide catheter and the orifice of the funnel may be expanded or collapsed via adjusting the tension on a wire controlling the size of the orifice. Alternatively, an outer catheter over the funnel is used to adjust the orifice. The funnel may include a pair of expandable rings of different diameter, or it may be configured from braided wires heat-shaped into a funnel. Preferably, a funnel sheath is provided. An engagement member may be provided on a support member.

### FIELD OF THE INVENTION

The present invention relates generally to catheters and related devices and methods of use thereof.

### BACKGROUND OF THE INVENTION

Removal of foreign bodies and thrombi, especially endovascular foreign bodies, using retrieval devices is frequently practiced during internal procedures, particularly endovascular procedures. In endovascular procedures, the process entails positioning of a guide catheter within a blood vessel. A retrieval device is introduced through the guide catheter and navigated toward the foreign body or lesion of interest in the vessel. The retrieval device is retrieved through the guide catheter once the foreign body or thrombus is captured.

One of the major challenges of the process is retaining the captured foreign body or thrombus within the retrieval device, particularly as the device is withdrawn from the vessel into the guide catheter. The narrower diameter of the guide catheter relative to the vessel creates difficulty in retaining the captured foreign body or thrombus within the retrieval device. The process may even be hazardous because the captured foreign body or thrombus can break away and result in occlusion of distal vessels.

A need exists for a device and related methods to facilitate easier removal of a foreign body or thrombus, especially devices and methods which enable retention of the captured foreign body or thrombus even as the retrieval device is retracted into the guide catheter.

### SUMMARY OF THE INVENTION

The present invention addresses the aforementioned challenge by providing a device that can be used as a guide catheter or a supplementary device that can be introduced through an outer catheter to facilitate the removal of foreign bodies or emboli by a retrieval device, or other internal devices. The supplementary device can be advanced to the site of reentry of the retrieval device into the outer catheter and then caused to assume a predetermined shape. By virtue of achieving a predetermined configuration having a three dimensional shape generally similar to a funnel or a portion of a cone, the device provides a safe reentry of the retrieval device into the guide catheter or outer catheter.

In one embodiment, a catheter device is provided. The catheter device includes a catheter with at least one end (which may be a distal end) configured to fit within a blood vessel and a funnel portion with a distal end and a proximal end. The proximal end of the funnel portion is affixed to the distal end of the catheter, and the distal end of the funnel portion has an opening with an adjustable or compressible diameter.

In another embodiment, a funnel device for a catheter for internal use is provided and has a funnel portion with a distal end and a proximal end. The proximal end of the funnel portion is configured to engage the distal end of a catheter and the distal end of the funnel portion has an opening with an adjustable diameter.

In yet another embodiment, a method of operating an internal device with a catheter is provided. The method involves positioning a catheter endovascularly and introducing the internal device through the catheter. Then the diameter of the end portion of the catheter is expanded to resemble a cone-shape and the internal device is retracted into the expanded end portion of the catheter. The method may also include the step of at least partly closing the open end of the cone-shape so as to retain any material collected by the internal device.

Other objects, advantages and novel features of the present invention will become apparent from the following detailed description of the invention when considered in conjunction with the accompanying drawings.

### BRIEF DESCRIPTION OF THE DRAWINGS

FIG. 1 depicts a lateral view of a funnel catheter device in accordance with one embodiment of the present invention;

FIG. 2 depicts a lateral view of the funnel catheter device shown in FIG. 1 but with the distal end of the funnel expanded;

FIG. 3 depicts a cross-sectional view of a guide catheter for use with the device shown in FIGS. 1 and 2;

FIG. 4 depicts a lateral view of a funnel catheter device in accordance with another embodiment of the present invention;

FIG. 5 depicts a lateral view of the funnel catheter device shown in FIG. 4 but with the distal end of the funnel expanded;

FIG. 6 depicts a lateral view of a funnel catheter device in accordance with still another embodiment of the present invention;

FIG. 7 depicts a lateral view of the funnel catheter device shown in FIG. 6, but with the distal end of the funnel expanded;

FIG. 8 depicts a lateral view of a funnel catheter device in accordance with yet another embodiment of the present invention;

FIG. 9 depicts a lateral view of the funnel catheter device shown in FIG. 8 but with the distal end of the funnel expanded;

FIG. 10 depicts a lateral view of a funnel catheter device in accordance with another embodiment of the present invention;

FIG. 11 depicts a lateral view of the funnel catheter device shown in FIG. 10 but with the distal end of the funnel expanded;

FIG. 12 depicts a lateral view of a funnel catheter device in accordance with another embodiment of the present invention; and

FIG. 13 depicts a lateral view of the funnel catheter device shown in FIG. 12 but with the funnel in a retracted position.

### DETAILED DESCRIPTION OF CURRENTLY PREFERRED EMBODIMENTS

Generally speaking, the funnel catheter of the present invention facilitates easy reentry of a device for internal use into a guide catheter by providing an enlarged orifice at the site of reentry. Suitable internal devices for use with certain embodiments of the invention specifically include known types of retrieval devices (e.g. snares or other grasping or retrieval devices).

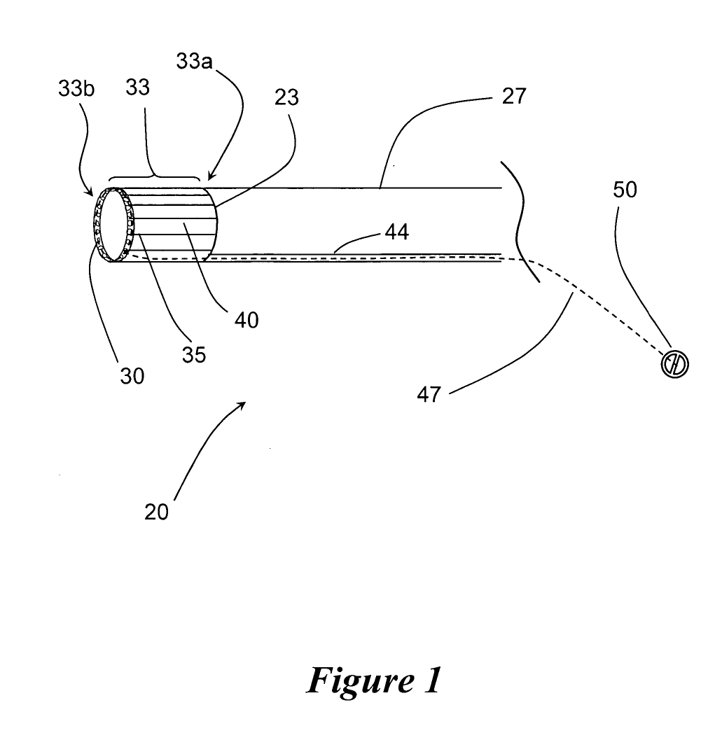

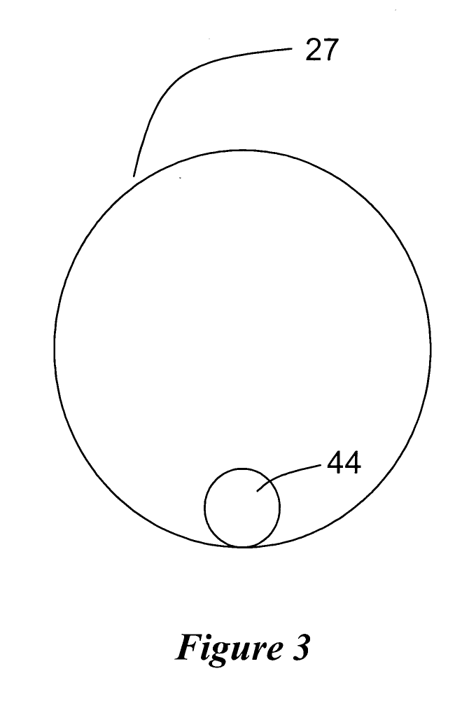

One embodiment of a funnel catheter device, designated generally by numeral 20, will be described below with reference to FIGS. 1, 2, and 3. A ring 23 is configured from nickel-titanium (NiTi) wire, or any other suitable material, preferably an alloy of nickel and titanium. Preferably, the material is about 0.004 inches in diameter. The ring 23 is attached at the proximal end 33a of a funnel 33. The proximal end 33a of the funnel 33 has a fixed diameter and is also attached to the distal end of a double-lumen guide catheter 27. Alternatively, the proximal end of the funnel 33 may be attached to the distal end of the guide catheter 27 without any ring 23.

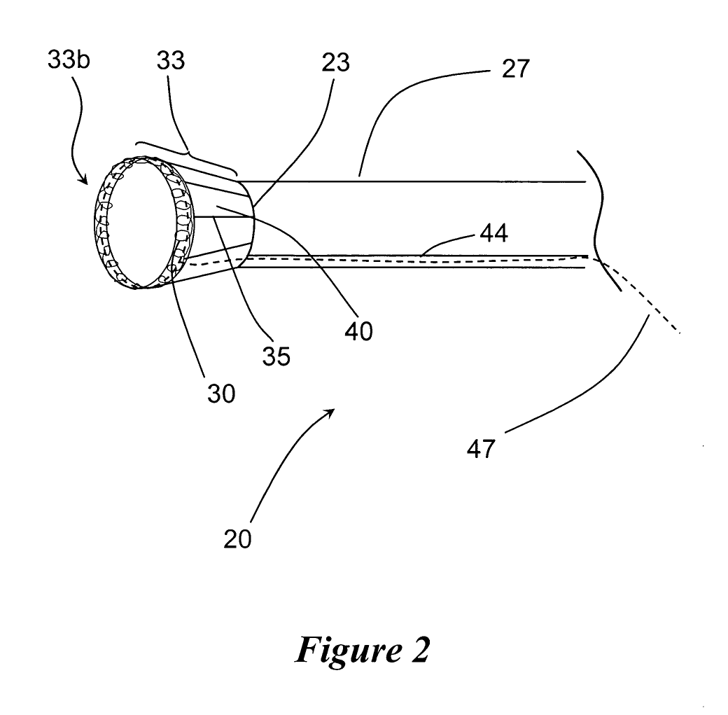

An expandable ring 30 is attached to the distal end 33b of the funnel 33. The expandable ring 30 is preferably a micro-coil ring and may be configured from NiTi wire, or any other suitable material. In a currently preferred embodiment, the expandable ring 30 is a hollow micro-coil ring and is configured from NiTi wire with a diameter of 0.004 inches in diameter. The expandable ring 30 is configured so that it can adapt to different diameter sizes as shown in FIGS. 1 and 2. A connective element 35, which may be configured from a plurality of wires, preferably NiTi wires, or any other suitable material, may be used to connect the ring 23 and the expandable ring 30 together. Where the connective element 35 is configured from a plurality of NiTi wires, the wires preferable have a diameter of at least 0.004 inches.

As used herein, the term micro-coil ring means a singular filament or wire composed of an alloy, especially a superelastic alloy such as NiTi, elgiloy, or another suitable metal. Preferably, the filament or wire can elongate at least up to five (5) percent of its length and is formed into a helical coil having an internal lumen or cavity, an outer diameter and a pitch formed by the helical arrangement. The internal lumen of the helical coil contains a separate filament called the control element 47 which may be a wire or configuration of wires composed of stainless steel, tungsten, NiTi or other metal to provide an actuating force which may be applied to the coil. Application of an actuating force to the coil causes the helix of the coil to contract or compress until the coil is held in a packed position with the adjacent wraps of the coil touching. The helical coil is generally configured as hoop or loop, the circumferential length of which is related to the compression state or degree of the coil. Thus, the overall diameter of the distal tip opening of the device may be changed by manipulating the compression of the coil with the control element 47. In this way, the opening of the distal end 33b of the funnel 33 may be actuated from a minimum diameter, at least partially closing the funnel 33, to a maximum diameter, opening the funnel 33.

The term funnel 33 is used herein to represent any device or configuration suitable for facilitating reentry of an internal device. The term funnel 33 is not intended to be and should not be interpreted to be limited to funnel-shaped devices, given that in certain embodiments of the invention, the outer opening of the funnel 33 is adjustable and may be adjusted such that the funnel more closely resembles a cylinder or another shape as is described below and is shown in at least FIGS. 1, 4, 6, 8 and 10. In this sense, the term funnel 33 refers to the characteristic feature of a device or configuration that allows it to assist with the proper positioning of a second device as it enters the funnel 33.

Control element 47, preferably configured as a wire or other cable, most preferably from NiTi, or any other suitable material, passes through the catheter inner lumen 44 and the expandable ring 30. The control element 47 preferably has a diameter at least 0.004 inches. Control element 47 is useful to adjust the tension on the expandable ring 30 and any associated funnel sheath 40. The expandable ring 30 and the control element 47 may be configured so that tension on the control element 47 exerts a compressive force about the expandable ring 30, thereby preventing the expandable ring from expanding. Release of tension on the control element 47 allows the expandable ring 30 to increase in diameter, thereby increasing the diameter of the opening on the distal end 33b of the funnel 33. Alternatively, the control element 47 may exert a compressive or an expansive force about the expandable ring 30, thereby causing the expandable ring 30 to decrease or increase in diameter.

The funnel sheath 40 is preferably about 0.003 inches in thickness and covers and partially makes up the funnel 33. Preferably the funnel sheath 40 is configured from expanded polytetrafluoroethylene (ePTFE), urethane, silicon, or another suitable polymer material. The funnel sheath 40 is preferably configured by rolling back ePTFE, or another suitable material, over itself to form multiple layers. Preferably at least the connective element 35 and the ring 23 are completely encapsulated between the layers. In certain embodiments, the funnel sheath 40 may be used without any connective element 35.

The tension on the control wire 47 and subsequently on the expandable ring 30 is adjusted via a knob 50, or other adjustment device, located at the proximal end of the guide catheter 27. This configuration allows the funnel 33 to open up at the distal end 33b upon reducing the tension on the expandable ring 30, to facilitate a smooth reentry of a retrieval device (not shown here). At the time of retrieval of the funnel catheter device 20 from the body, the tension on the control wire 47 may be adjusted such that the diameter of the expandable ring 30 at the distal end 33b of the funnel 33 matches or nearly matches the diameter of the guide catheter 27.

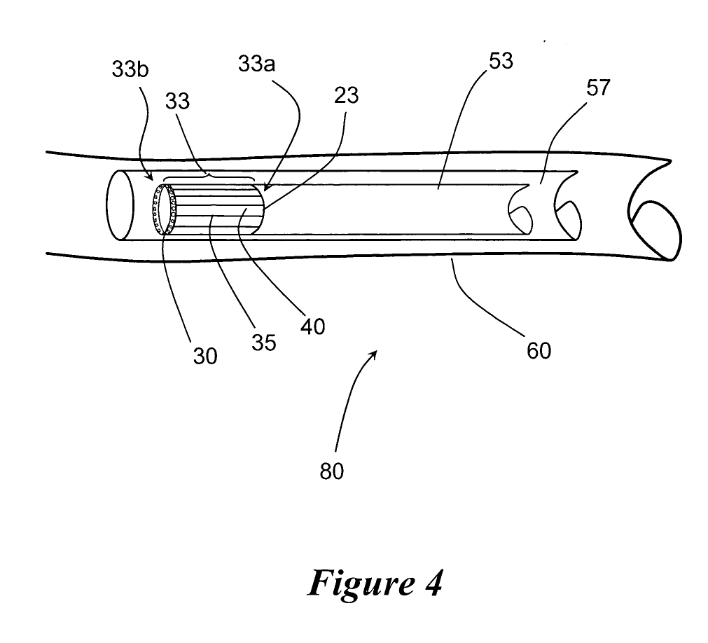

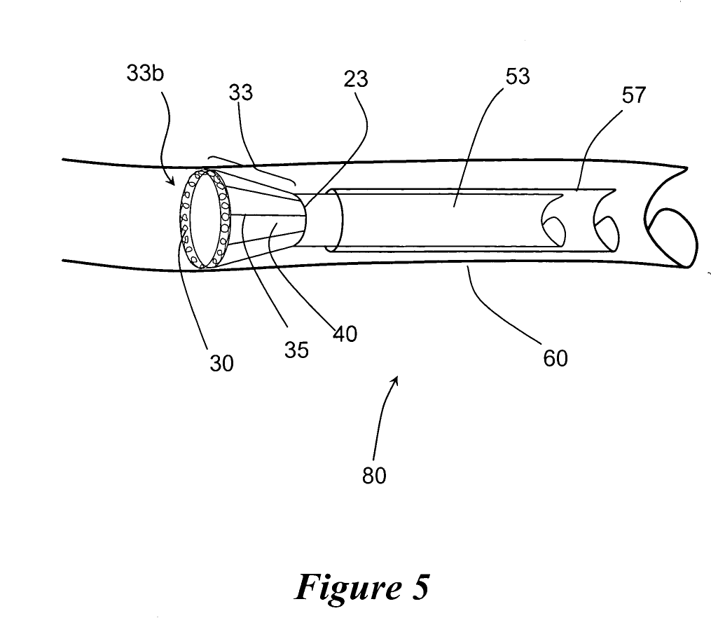

FIGS. 4 and 5 show another embodiment of the present invention designated generally by numeral 80. Vessel 60 is shown in FIGS. 4 and 5 for purposes of clarity only and vessel 60 is not intended as and should not be interpreted to be a required part of the present invention. This is true for the numerous instances of vessel 60 in the figures herewith.

A ring 23 is configured from NiTi wire, or any other suitable material. In a preferred embodiment the ring 23 is configured from NiTi wire with a diameter of about 0.004 inches. The ring 23 is attached to the proximal end 33a of a funnel 33 and the proximal end 33a of the funnel 33 has a fixed diameter. The distal end 33b of the funnel 33 includes an expandable ring 30 configured from NiTi wire, or another suitable material. In a currently preferred embodiment, the expandable ring is configured from NiTi wire with a diameter of about 0.004 inches. The expandable ring 30 is configured to provide an opening with an adjustable or compressible diameter.

The ring 23 at the proximal end 33a and the expandable ring 30 at the distal end 33b of the funnel 33 may be connected together via a connective element 35. In a currently preferred embodiment the connective element 35 is configured as a plurality of wires. These wires of the connective element 35 are preferably configured from NiTi, having a diameter of about 0.004 inches. The wires of the connective element 35 may also be configured from other suitable materials and in other suitable sizes.

The funnel 33 is covered with a funnel sheath 40 configured as described above. The proximal end 33a of the funnel 33 may be attached directly to the distal end of the guide catheter 53, without the use of a ring 23. The opening provided at the distal end 33b of the funnel 33 is contained by an outer shaft 57 over the guide catheter 53. This outer shaft 57 is preferably configured so that it prevents expansion of the expandable ring 30 when the expandable ring 30 is within the outer shaft 57, as shown in FIG. 4.

Once the guide catheter 53 is positioned in a vessel 60, the outer shaft 57 may be removed from the end of the guide catheter 53, either fully or partially, allowing the distal end 33b of the funnel 33 to be opened, as shown in FIG. 5. This facilitates a smooth reentry of the retrieval device (not shown). The distal end 33b of the funnel 33 and the expandable ring 30 may be compressed by the outer shaft 57 by repositioning the outer shaft at least partially over the distal end 33b of the funnel 33. Although not shown, it can be appreciated from FIG. 5 by one of ordinary skill in the art that positioning the outer shaft 57 partially over the funnel 33 will cause a corresponding reduction in the diameter of the expandable ring 30 and the distal end 33b of the funnel 33. Further, the outer shaft may be repositioned entirely over the distal end 33b of the funnel 33 and the expandable ring 30, thereby compressing the expandable ring 30 and the attached distal end 33b of the funnel 33 to the diameter of the outer shaft 57. In this way, the diameter of the expandable ring 30 may be adjusted as necessary to avoid damage to the vessel 60 during retrieval of the guide catheter 53.

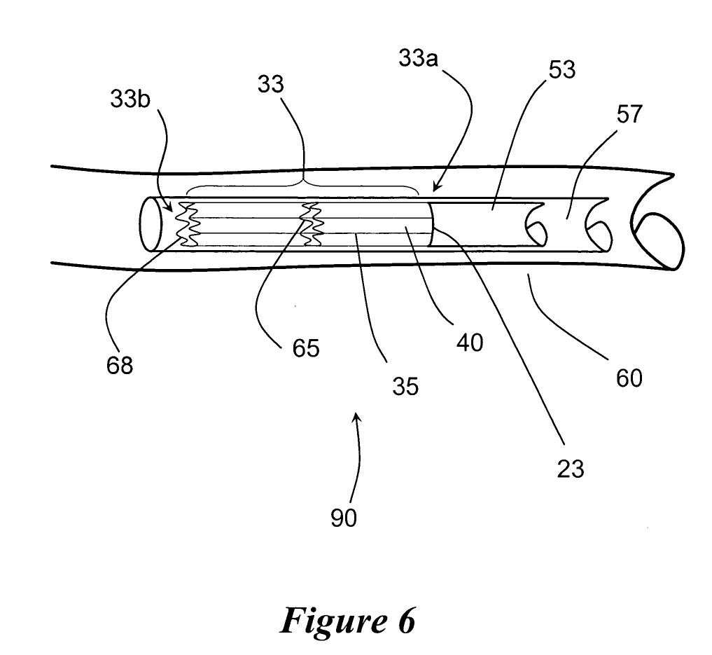

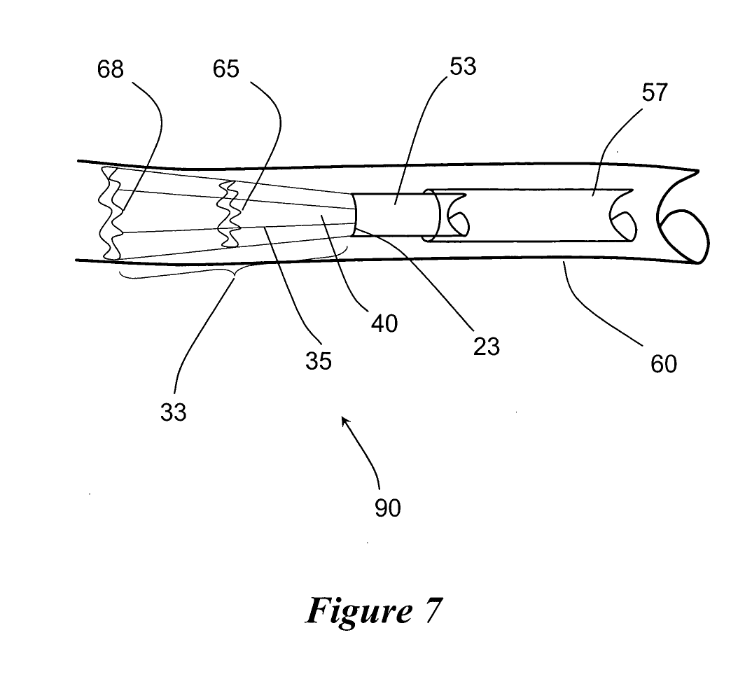

FIGS. 6 and 7 show another embodiment of a funnel catheter device, designated generally by numeral 90. The funnel 33 is configured with a ring 23 at the proximal end 33a. The ring 23 is preferably configured from NiTi wire, or some other suitable material, having a diameter of about 0.004 inches. The proximal end 33a of the funnel 33 has a fixed diameter and is attached to the distal end of a guide catheter 53.

The funnel 33 includes a first expandable ring 65 and a second expandable ring 68. The first expandable ring 65 is provided nearer the proximal end 33a of the funnel 33, and the second expandable ring 68 is provided nearer or at the distal end 33b of the funnel 33. Preferably the first expandable ring 65 and the second expandable ring 68 each have a different diameter when fully expanded. Both the first expandable ring 65 and the second expandable ring 68 may be configured from heat-shaped Nitinol wire with a diameter of at least 0.004 inches. Alternatively, one or both of the first expandable ring 65 and the second expandable ring 68 may be configured from another suitable material, and optionally differently sized, material. Further, one or both of the first expandable ring 65 and the second expandable ring 68 may be configured as a zigzag ring, wherein a cross-section of the ring itself has a shape resembling a wave, for instance a sine curve.

The first expandable ring 65 and the second expandable ring 68 are connected to the distal end of the guide catheter 53 by a connective element 35, which may be configured from a plurality of NiTi wires that are distributed evenly in the radial direction. Where the connective element 35 is configured from a plurality of wires they are preferably uniform in size and are covered with a funnel sheath 40 as described above. Alternatively, the first expandable ring 65 and the second expandable ring 68 may be connected without a connective element 35, using simply a funnel sheath 40.

The proximal end 33a of the funnel 33 is bonded to the distal end of the guide catheter 53. The distal end 33b of the funnel 33 is contained by an outer shaft 57 over the guide catheter 53. Once the guide catheter 53 is positioned in the vessel 60, the outer shaft 57 may be moved, allowing the distal end 33b of the funnel 33 to be opened. This facilitates a smooth reentry of the retrieval device (not shown here). The distal end 33b of the funnel 33 may then be compressed when the outer shaft 57 is positioned back over the distal end 33b of the funnel 33, thereby avoiding damage to the vessel 60 during the retrieval of the guide catheter 53. Thus, similar to the embodiment depicted for FIGS. 4 and 5, the opening provided by the first expandable ring 65 and the second expandable ring 68 can be adjusted by moving the outer shaft 57 laterally along the catheter 53.

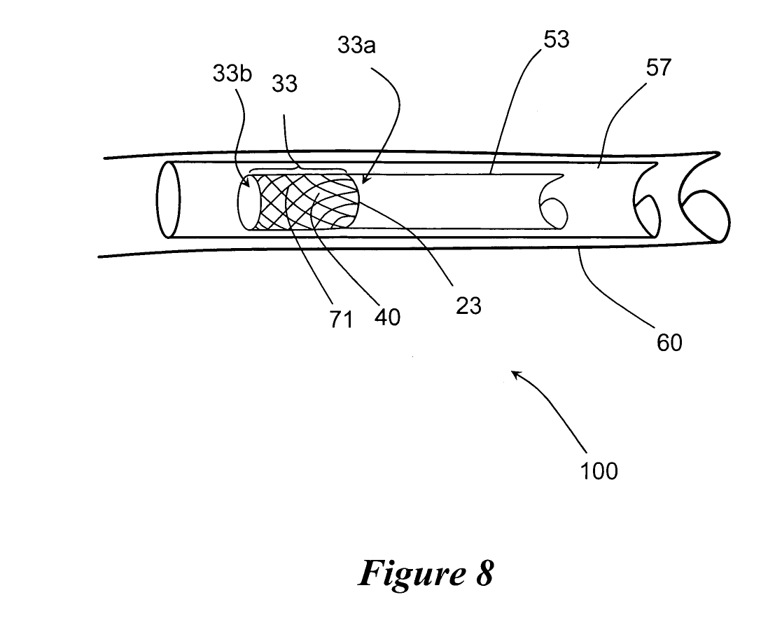

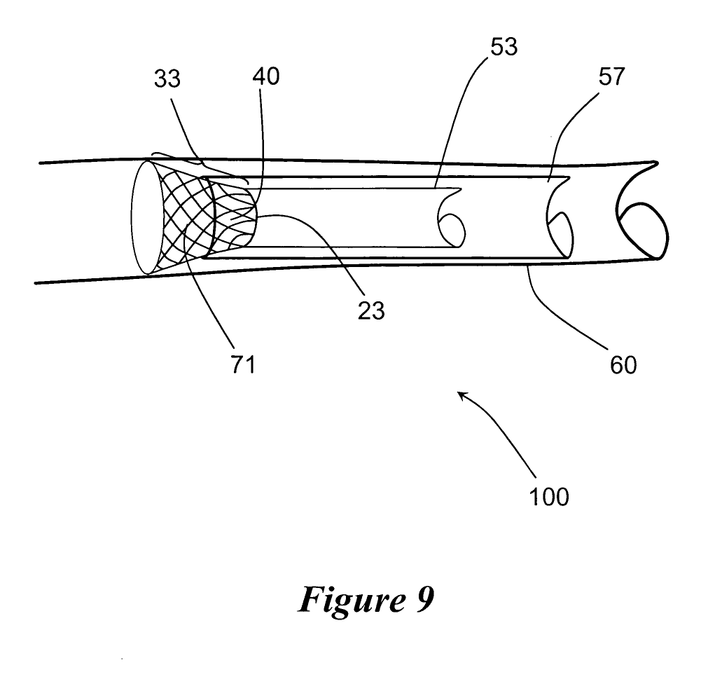

FIGS. 8 and 9 depict another embodiment of the present invention, designated generally by numeral 100. In this embodiment, the funnel 33 is configured from a flexible support material 71. Preferably, the support material 71 is configured as or from a plurality of braided support elements, and in one embodiment these support elements are wires with a diameter of about 0.004 inches although they may optionally be made in other suitable sizes. These support elements are preferably configured from NiTi; however, they may also be configured from other suitable materials. The proximal end 33a of the funnel 33 has a fixed diameter and is attached to the distal end of a guide catheter 53.

In certain embodiments, the support material 71 may be covered with a funnel sheath 40 such as that previously described.

The distal end 33b of the funnel 33 is contained by an outer shaft 57 over the guide catheter 53. Once the guide catheter 53 is positioned in the vessel 60, the shaft 57 may be removed, thereby allowing the distal end 33b of the funnel 33 to expand. This facilitates a smooth reentry of the retrieval device (not shown). The distal end 33b of the funnel 33 may then be compressed when the outer shaft 57 is positioned back over the distal end 33b of the funnel 33, thereby avoiding damage to the vessel 60 during the retrieval of the guide catheter 53.

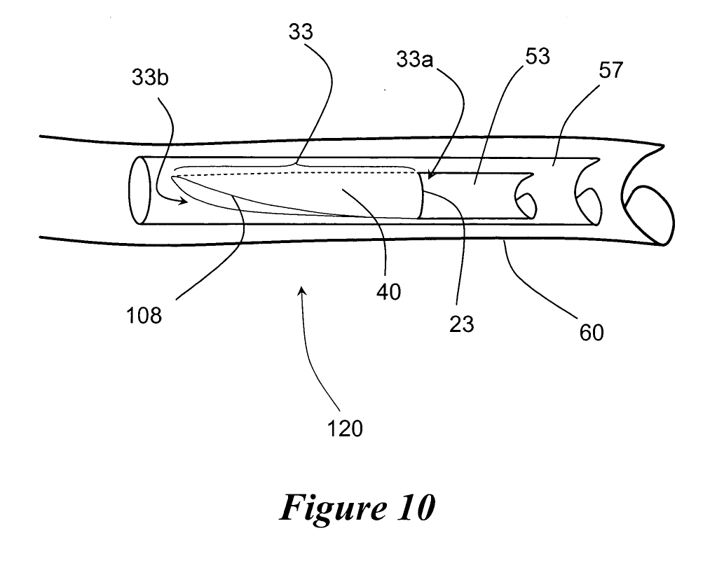

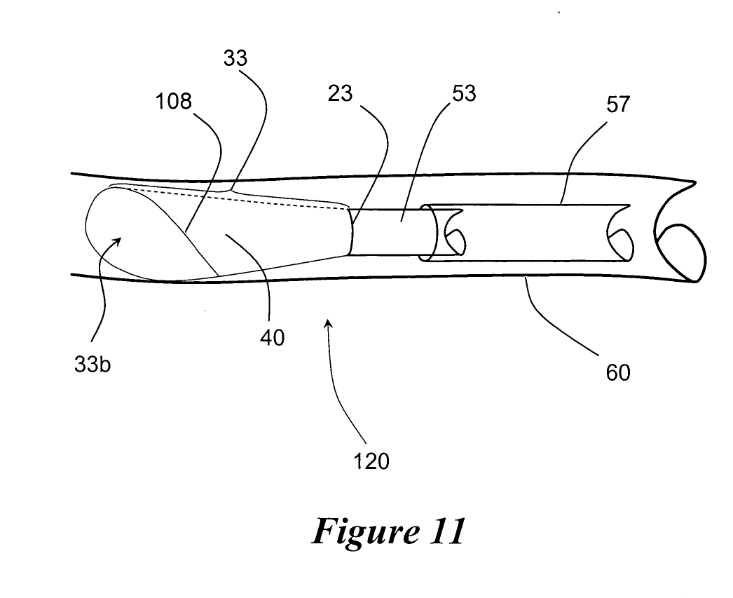

FIGS. 10 and 11 show another embodiment of the present invention, designated generally by numeral 120. A loop 108 is made from NiTi wire, or any other suitable material. In a preferred embodiment, the loop 108 is configured from NiTi wire with a diameter of about 0.004 inches and is heat shaped into a loop as shown in FIGS. 10 and 11. The loop 108 is covered with a funnel sheath 40 configured as described above. Together the loop 108 and the funnel sheath 40 make up a funnel 33. The funnel sheath 40 is preferably about 0.003 inches in thickness and covers and partially makes up the funnel 33. Preferably the funnel sheath 40 is configured from ePTFE or urethane or another suitable material. The funnel sheath 40 is preferably configured by rolling back ePTFE, or another suitable material, over itself to form multiple layers.

The expandable nature of the loop 108 is such that when no external pressure is applied it expands into a shape generally resembling that shown in FIG. 11. The proximal end 33a of the funnel 33 may be attached directly to the distal end of the guide catheter 53. The opening provided by the expandable loop 108 at the distal end 33b of the funnel 33 may be contained by an outer shaft 57 over the guide catheter 53. This outer shaft 57 is preferably configured so that it prevents expansion of the loop 108 when the loop 108 is within the outer shaft 57, as shown in FIG. 10.

Once the guide catheter 53 is positioned in a vessel 60, the outer shaft 57 may be removed from the end of the guide catheter 53, either full or partially, allowing the distal end 33b of the funnel 33 to be opened, as shown in FIG. 11. This facilitates a smooth reentry of the retrieval device. Further, the outer shaft 57 is preferably positioned during removal of the guide catheter 53 so that the outer shaft 57 causes at least a partial closure of the loop 108 at the distal end 33b of the funnel 33. This helps to avoid damage to the vessel 60 during removal of the guide catheter 53.

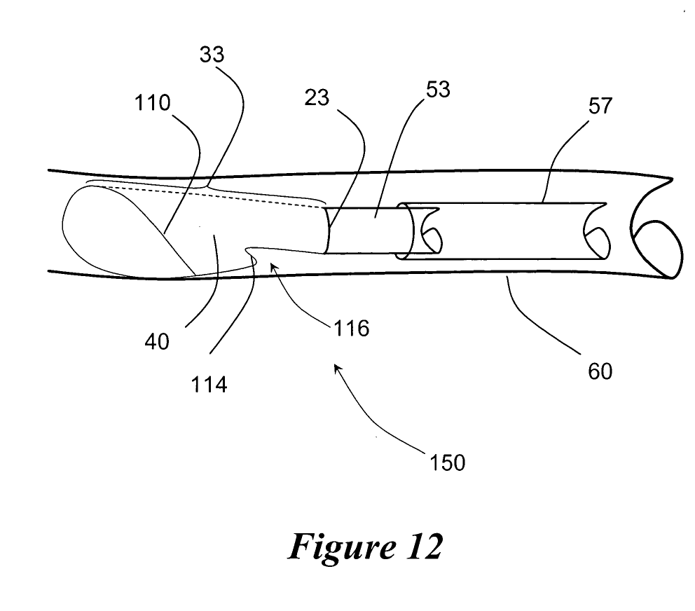

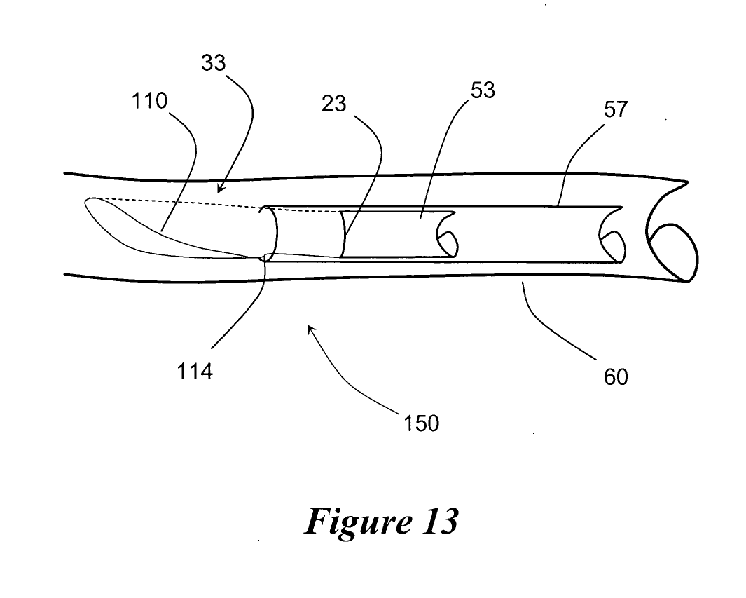

FIGS. 12 and 13 show another embodiment of the present invention, designated generally by numeral 150. The embodiment of the device 150 shown in FIGS. 12 and 13 is similar to the device 120 shown in FIGS. 10 and 11. A loop 110 is made from NiTi wire, or any other suitable material. In a preferred embodiment, the loop 110 is configured from NiTi wire with a diameter of about 0.004 inches and is heat shaped into a loop as shown in FIGS. 10 and 11.

The loop 110 is provided with a support member 116 which may connect the loop 110 to the guide catheter 53. The support member 116 is provided with an engagement member 114 which serves to engage the outer shaft 57 when the guide catheter 53 is moved laterally in relation to the outer shaft 57. In the embodiment depicted in FIGS. 12 and 13, the engagement member 114 is a curve in the support member 116. Engagement member 114 is not limited, however, to being a curve and may be provided in a variety of different configurations or embodiments, as would be appreciated by one skilled in the art. For instance, the engagement member 114 may be a loop or simply a bulge in the support member 116. In yet another embodiment (not shown) the engagement member 114 may be a separate attachment to the support member 116. In such an arrangement, the engagement member 114 may be clamped or otherwise attached to the support member 116 using commonly known attachment methods.

In operation, the engagement member 114 acts as a fulcrum or lever arm to actuate the at least partial closing of the loop 110 before the loop 110 is pulled into the outer shaft 57. As a result, the funnel 33 may be maintained in an at least partially closed arrangement. Without the engagement member 114, the funnel 33 would close as it is moved laterally in relation to and inside of the outer shaft 57. In certain instances such closure may not be desirable as some material within the funnel 33 may be lost as the funnel 33 or the loop 110 changes its shape. Thus, the engagement member 114 provides a way to facilitate the capture of more of the contents of the funnel 33 by closing the loop 110 which serves as the opening of the funnel 33 before the funnel 33 is brought into the outer shaft 57.

In another embodiment of the invention, a funnel device for a catheter for internal use is provided. Such a funnel device may take on a variety of forms, including any of those shown for funnel 33 in FIGS. 1-9. In particular, the funnel device preferably includes a funnel portion having a distal end 33b and a proximal end 33a. The proximal end 33a is configured to engage the end of a catheter such as catheter 27 of FIG. 1, and the distal end 33b has an opening with an adjustable diameter.

The funnel device may be provided with a ring 23 which defines the proximal end of the funnel device and an expandable ring 30 which defines the distal end of the funnel portion. In one embodiment, the expandable ring 30 is a hollow micro-coil ring, as described above.

The funnel device may also include a funnel sheath 40 as described above, which encapsulates at least part of the funnel 33. Optionally, the funnel device may include a connective element 35, which may be configured from a plurality of wires, which connects the ring 23 and the expandable ring 30.

In another embodiment, such as that shown in FIGS. 6 and 7, the funnel device includes a first expandable ring 65 and a second expandable ring 68. In such an embodiment, the first expandable ring 65 and second expandable ring 68 may have differing diameters. Further, the expandable ring may be a zigzag ring, as described above.

In another embodiment, such as that shown in FIGS. 8 and 9, the funnel 33 of the funnel device is made from a support material 71. As described above, this support material may be configured from a plurality of braided support elements.

In another embodiment, such as that shown in FIGS. 10 and 11, the distal end of the funnel 33 is defined by a loop 108. The loop 108 is preferably heat shaped into a loop and configured from a material that possess a high shaped memory property, i.e., the loop 108 prefers to retain or return to its preset shape, even after being significantly bent or distorted. Preferably, the loop 108 is compressible.

In another embodiment, such as that shown in FIGS. 12 and 13, the funnel 30 is provided with a support member 116 having an engagement member 114. The engagement member 114 serves as a lever or fulcrum to facilitate at least partial closing of the distal end of the funnel 30.

The present invention also includes a method for operating an internal device with a catheter. The method includes the step of positioning a catheter internally in the body or positioning a catheter within a vessel. Thereafter, the internal device is introduced through the catheter and into the patient, whereupon, the diameter of an end portion of the catheter is expanded. Preferably the diameter is expanded into a three-dimensional shape resembling a funnel-shape or at least part of a cone. For purposes of the present disclosure, the term “expanding the diameter of an end portion of the catheter into a funnel-shape” simply means expanding the diameter of an end portion of the catheter so that the diameter is increased relative to the diameter before the expansion.

Finally, the internal device is retracted into the expanded end portion of the catheter, so as to facilitate safe and easy removal of the internal device and catheter.

In a currently preferred method, the step of expanding the diameter of an end portion of the catheter includes expanding an expandable ring affixed to an end portion of the catheter by adjusting a control element associated with the expandable ring. This adjustment may include, for instance, increasing or decreasing tension on the control element.

In another currently preferred method, the step of expanding the diameter of an end portion of the catheter includes repositioning an outer shaft to at least partially release an end portion of the catheter to allow the diameter of an end portion of the catheter to expand.

The method may also include the step of at least partially closing an opening provided at an end portion of the catheter. In such a method, the distal end of the funnel portion of the device is such that it may be at least partially closed, so as to increase the likelihood of retaining all the material collected by the internal device. Thus, this step is preferably performed once the internal device is retracted into the funnel portion of the device.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof. In particular, except where specifically noted, the claims are not limited to the materials and/or dimensions described in the specification, since these materials and/or dimensions are merely provided to describe features of particular embodiments of the invention. One skilled in the art would easily appreciate that other materials may be substituted and other dimensions used, without departing from the true spirit and substance of the present invention.

1. A device comprising:
a catheter with a distal end configured to fit within a vessel and
a funnel portion having a distal end and a proximal end, said proximal end of the funnel portion being affixable to said distal end of the catheter and said distal end of the funnel portion having an opening with an adjustable diameter.

2. The device of claim 1, wherein said funnel portion comprises a ring which defines the proximal end of the funnel-shaped portion which comprises at least one expandable ring defining the distal end of the funnel portion.

3. The device of claim 2, wherein said at least one expandable ring is a hollow micro-coil ring.

4. The device of claim 2, further comprising:
an inner lumen associated with the catheter, a control element housed at said inner lumen, a funnel-shaped portion having a distal end with an adjustable opening and a proximal end being affixable to an end of said catheter, said control element being operatively associated with said expandable ring such that the diameter of said opening on said distal end is controlled by the control element.

5. The device of claim 4, further comprising an adjustment device configured to facilitate adjustment of tension on said control element.

6. The device of claim 1, further comprising a sheath which encapsulates said funnel portion.

7. The device of claim 2, further comprising an element connecting the ring and the expandable ring.

8. The device of claim 7, wherein said element comprises a plurality of wires.

9. The device of claim 2, further comprising an outer shaft which is configured to at least partially house the said catheter, and be slidably positionable at least along the catheter.

10. The device of claim 9, wherein said expandable ring is configured to expand to a diameter provided by said outer shaft or a vessel, and said outer shaft is slidably positionable along the catheter and said funnel-shaped portion to adjust a diameter of the opening provided by said distal end of the funnel-shaped portion.

11. The device of claim 2, wherein said at least one expandable ring comprises a first expandable ring and a second expandable ring.

12. The device of claim 11, wherein said first expandable ring and said second expandable ring have different diameters.

13. The device of claim 2, wherein said at least one expandable ring comprises a zigzag ring.

14. The device of claim 1, wherein said funnel-shaped portion comprises a support material.

15. The device of claim 14, wherein said support material comprises a plurality of braided support elements.

16. The device of claim 1, further comprising means for expanding a diameter of said opening on said distal end of said funnel-shaped portion.

17. A device for a catheter comprising:
a funnel-shaped portion having a distal end and a proximal end, said proximal end being configured to engage the end of the catheter and said distal end having an opening with an adjustable diameter.

18. The device of claim 17, wherein said funnel-shaped portion comprises a ring which defines the proximal end thereof and an expandable ring which defines the distal end thereof.

19. The device of claim 18, wherein said expandable ring is a hollow micro-coil ring.

20. The device of claim 17, further comprising a funnel sheath which encapsulates said funnel-shaped portion.

21. The device of claim 18, further comprising a plurality of wires connecting the ring and the expandable ring.

22. The device of claim 17, wherein said funnel-shaped portion includes a first expandable ring and a second expandable ring.

23. The device of claim 22, wherein said first expandable ring and said second expandable ring have different diameters.

24. The device of claim 18, wherein said expandable ring comprises a zigzag ring.

25. The device of claim 17, wherein said funnel portion comprises a support material.

26. The device of claim 25, wherein said support material comprises a plurality of braided support elements.

27. The device of claim 17, wherein said distal end is at least partly defined by a heat-shaped ring.

28. The device of claim 27, wherein said heat-shaped ring is compressible.

29. The device of claim 27, further comprising a funnel sheath.

30. The device of claim 17, further comprising a support member provided with an engagement member.

31. The device of claim 30, wherein said engagement member facilitates at least partial closing of the distal end of the funnel-shaped portion.

32. A method of operating an internal device with a catheter comprising:
positioning a catheter within a patient's body;
introducing the internal device through said catheter;
expanding a diameter of an end portion of the catheter into a funnel shape; and
retracting the internal device into the expanded end portion of the catheter.

33. The method of claim 32, wherein said expanding comprises expanding a ring affixed to the end portion of the catheter by adjusting a control element associated with the ring.

34. The method of claim 32, wherein said expanding comprises repositioning an outer shaft to at least partially release the end portion of the catheter and to allow a diameter of said end portion of the catheter to expand.

35. The method of claim 32, further comprising the step of at least partially closing an opening provided at the end portion of the catheter.
